(19) United States
(12) Patent Application Publication
(10) Pub. No.: US 2011/0171656 A1
(43) Pub. Date: Jul. 14, 2011

(54) METHOD FOR NORMALIZING THE CONTENTS OF BIOMOLECULES IN A SAMPLE

(75) Inventors: Christoph Erbacher, Haan (DE); Peter Grünefeld, Hilden (DE)

(73) Assignee: Qiagen GmbH

(21) Appl. No.: 13/063,598

(22) PCT Filed: Sep. 16, 2009
(86) PCT No.: PCT/EP2009/061990

§ 371 Date: Mar. 11, 2011

(30) Foreign Application Priority Data
Sep. 17, 2008 (DE) 102008047790.7

(51) Int. Cl. C12Q 1/68; G01N 33/53

(52) U.S. Cl. 435/6.12; 436/501; 435/7.1; 435/7.92; 422/68.1

(57) ABSTRACT

The present invention relates to a method for normalizing the contents of biomolecules in a sample, comprising the following steps: a) preparing a reaction vessel with a vessel surface that is functionalized at least in sections—preferably on the inside of the vessel—in such a way that the surface can reversibly bind biomolecules under high salt conditions, b) executing at least one sample preparation step, c) binding biomolecules from the prepared sample to the vessel surface (“binding and normalizing step”) under high salt conditions, d) optionally washing (“washing step”), and e) executing at least one subsequent reaction. The application also relates to a reaction vessel as is used in the above method.

### FIELD OF THE INVENTION

The present invention relates to a method, a use, and an apparatus for normalizing the content of biomolecules in a sample. The method, the use, and the apparatus are suitable for applications in biochemistry, molecular biology, molecular genetics, microbiology, molecular diagnostics, and/or molecular forensics, for example.

### TECHNICAL BACKGROUND

Normalizing the content of biomolecules in a sample plays a major role in the analysis of samples, for example in molecular diagnostics, for gene expression analysis, in active substance-based transcript level analysis, molecular forensics, sequencing, or genotyping.

The reason for normalization is that in some cases the biomolecules to be detected, in particular nucleic acids and/or proteins, may be present in different quantities in the sample. In addition, the preparative sample processing steps, for example lysis, cell denaturation, an isolation step, or reverse transcription, may provide the biomolecules in question at different levels of efficiency.

In both cases, this means that the unknown portion of biomolecules in the sample due to the above-mentioned uncertainty factors complicates the reproducibility and the accuracy of the further preparation and analysis steps, or necessitates additional steps for the quantification.

This is particularly important for the preparation of samples containing nucleic acid which are to be subsequently detected using known methods (polymerase chain reaction (PCR), reverse transcription, immuno-PCR). In this case it is important to coordinate the process parameters, in particular the content of biomolecules in the sample, in order to achieve a desired number of cycles.

The polymerase chain reaction (PCR) has become established in the past few decades as a system for the in vitro amplification of DNA. At the same time, PCR has become a widely used analytical and assay tool as the result of further development of the technique from mere duplication toward qualitative analysis and “real-time” PCR for quantification. The detection of so-called messenger RNA (mRNA), the RNA transcripts of certain gene segments, is currently a standard application. In this regard, all of the mRNA present in a sample is transcribed back to DNA (cDNA) using a primer (frequently poly dT) and an enzyme (reverse transcriptase), and the cDNA thus formed is detected in a PCR assay. It is thus possible to carry out the assays in a one- or two-step protocol. In the one-step protocol, the reverse transcription (RT) as well as the PCR assay itself is performed in the PCR instrument, whereas in the two-step protocol the reverse transcription is performed separately and an aliquot of the reaction is added to a PCR mastermix. However, the mRNA level may be very different for different samples, and in addition the efficiency of the RT may be subject to fluctuations. This sometimes results in very different quantities of cDNA which are introduced into the PCR reaction.

In the prior art, as a rule the RNA content of a sample is determined before starting the reverse transcription, for example with the aid of OD measurements or fluorescence measurements. Quantification on the cDNA level is generally not performed, since the presence of nucleotides, ribosomal RNA, and other constituents after completion of reverse transcription complicates quantification of the cDNA.

Such a quantification approach may be implemented in particular in the so-called two-step method, in which the sample preparation (by lysis, cell denaturation, isolation step, or reverse transcription, for example) and the further sample processing (by PCR, for example) are carried out in separate steps and/or in different vessels, so that a pipetting step may be performed between the two steps. Such a step is also suitable for methods in which a sample which may already be prepared is directly subjected to a PCR process.

For the case of the two-step PCR from the prior art, the reverse transcription is performed in a separate vessel, and pipetting to a PCR mastermix is then carried out. At this point the quantity of cDNA after the reverse transcription could be quantified one time spectroscopically. However, this is usually dispensed with due to the additional level of effort, and only an aliquot of the RT reaction is used in the qPCR. To prevent excessive dilution of the PCR mastermix, only a maximum of 10% of the mastermix quantity should be added thereto as aliquot. This means that in a 25-μL PCR using 2.5 μL, only slightly more than 10% of the product of the reverse transcription may be used in a PCR reaction, or once again larger volumes must be avoided. At the same time, the additional pipetting step results in further inaccuracy, as is the case for any manual operation. There is also an additional risk of cross-contamination.

In the present method it is not necessary to take additional volume in the PCR reaction assay into account, since the cDNA is reversibly bound to a defined surface in the reaction vessel.

The previous approach is not practicable for methods in which the sample preparation (by lysis and/or reverse transcription, for example) and the further sample processing (by PCR, for example) occur in the same vessel, and the vessel is to remain closed if possible (so-called one-step method).

In one-step methods from the prior art, a quantity of biomolecules (mRNA, for example) is used which is optionally spectroscopically quantified. The reverse transcription as well as the PCR method itself is carried out in the same reaction vessel. Thus, since the reaction vessel is not opened, if possible, during the process, the entire assay of the reverse transcription is transferred to the PCR reaction. For the samples used, however, the biomolecule content (of mRNA, for example) may be very different, and the efficiency of the sample preparation step (the reverse transcription, for example) is also subject to fluctuations, which sometimes results in very different product quantities after completion of the sample preparation step (cDNA, for example), which are then introduced into the subsequent reaction (PCR, for example), resulting in nonreproducible reaction results. Due to the additional reaction assay volume of typically 20 μL, in addition it is not possible to operate with a volume of 25 μL, which is customary for PCR. As a rule, one-step RT-PCRs are carried out on a 50-μL or 100-μL scale.

In the method according to the invention and the apparatus according to the invention, an absolute quantity of nucleic acid, for example, is normalized in an apparatus, using a binding surface produced according to the invention, instead of quantifying same using OD measurements or fluorescence measurements. In contrast, the so-called “housekeeper approach” represents an internal or endogenous reference which may also be used to make a statement concerning the state of the biological system. The quality of the assay may be assessed using the housekeeper gene, since for certain housekeeper genes standard values exist for their expression level in conjunction with certain cell types.

However, it must be noted that in this approach the accuracy of the normalization is greatly limited due to possible uncertainties. Thus, there is no ideal housekeeper gene; i.e., in any case a species-, type-, stage-, or state-specific variance of the gene expression may be observed which cannot be completely eliminated, even by the use of multiple housekeeper genes.

In addition, a method is known from US 20070231892 for amplifying nucleic acids, wherein in a vessel a lysate of a biological sample is contacted with a so-called “charge-switch” surface in order to bind the nucleic acids contained in the lysate. The unbound lysate is then removed, and the bound nucleic acids are amplified. For the referenced “charge-switch” material a change in the surface charge occurs when the pH changes. This property of weak ion exchangers appears, for example, at a pH which is below the pKs value of the surface groups, so that these surface groups have a positive surface charge. Negatively charged biomolecules, in particular nucleic acids, may then be bound. On the other hand, for a pH value above the pKs value of the surface groups the charge changes from positive to neutral or negative, so that negatively charged biomolecules, in particular nucleic acids, may once again be released. Thus, by using suitable buffers which have various pH values and which also have low salt concentrations (“low salt buffers”), the binding and release process may thus be controlled via the pH.

The referenced “charge-switch” material has anion exchanger properties in particular. One disadvantage of this approach is that suitable materials cannot be permanently attached covalently, for example, on the surface of microreaction vessels made of polypropylene, for example. The materials are generally immobilized by simple stacking on the surfaces. However, permanent adhesion to the surfaces is not ensured, which calls into question the reproducibility of these methods, and also makes multiple use of the correspondingly coated vessels impossible.

In addition, the isolation of RNA from biological samples under the referenced conditions, using “charge-switch” materials and generally using anion exchangers, is problematic due to the ubiquitous RNases. These remain intact under the prevailing low salt conditions, so that RNA is severely degraded within a few seconds, and detection is made more difficult or even impossible.

### DEFINITIONS

The term “nucleic acid” within the meaning of the present invention is understood to mean in particular, but is not limited to, natural, preferably linear, branched, or circular, nucleic acids such as RNA, in particular mRNA, single-strand and double-strand viral RNA, siRNA, miRNA, snRNA, tRNA, hnRNA, or ribozymes, genomic, bacterial, or viral DNA (single-strand and double-strand), chromosomal and episomal DNA, freely circulating nucleic acid, and the like, synthetic or modified nucleic acids, for example plasmids or oligonucleotides, in particular primers, probes, or standards used for the PCR, nucleic acids labeled with digoxigenin, biotin, or fluorescent dyes, or so-called “peptide nucleic acids” (PNAs).

The term “normalizing the content of biomolecules in a sample” is understood to mean a step which ensures that the content of biomolecules in the sample does not exceed a specified degree (according to the invention, by means of the size and the binding properties of at least a portion of the vessel surface, preferably at the inside of the vessel). This is a method for quantifying the content of biomolecules to a specified value. This includes subsequent discarding of biomolecules which exceed this degree, and also means that if the sample contains fewer biomolecules than the specified degree described above, the normalization is not successful.

The term “immobilization” within the meaning of the present invention is understood to mean in particular, but is not limited to, a reversible immobilization on a suitable solid phase.

The term “membranes” is understood to mean in particular, but is not limited to, solid phases which are able to reversibly bind biomolecules.

The term “high salt buffer” is understood to mean in particular, but is not limited to, a buffer having a high salt concentration (preferably chaotropic substances), preferably ≧100 mM, more preferably ≧500 mM, and even more preferably ≧1 M.

The term “high salt conditions” below is understood to mean an environment which uses a high salt buffer, preferably a high salt buffer containing chaotropic salts. The solubility of nucleic acids in water is reduced by using high salt [buffers], preferably containing chaotropic salts. The reason is the breaking down of hydrogen bridges and the associated decrease in the stability of secondary and tertiary structures of the nucleic acids in water. If a polar surface is then provided as a hydrogen bridge donor, the nucleic acids bind to this surface, since at that location they are more stable than in water. If the salt concentration is decreased, water once again becomes a better hydrogen bridge donor than the polar surface, and the nucleic acids may be detached from the surface.

The term “chaotropic substances” or “chaotropic salts” is understood to mean in particular, but is not limited to, substances which alter the secondary, tertiary, and/or quaternary structure of proteins and/or nucleic acids, and which leave at least the primary structure intact, which reduce the solubility of polar substances in water, and/or which intensify hydrophobic interactions. Preferred chaotropic substances are guanidine hydrochloride, guanidinium (iso)thiocyanate, sodium iodide, sodium perchlorate, potassium iodide, sodium (iso)thiocyanate, and/or urea.

The term “silanol groups” is understood to mean in particular, but is not limited to, silicon oxide (amorphous, crystalline) or polysilicic acid having the composition (SiO₂ₓ(OH)_{α}(OEt)_{β}, where the stoichiometric factor a is a function of x and β (i.e., α=4(1−x)−β). Silicon dioxide or polysilicic acid may contain one or more of the following substituents, or may also be completely replaced by the following oxides:

B₂O₃ (0-30%),

Al₂O₃ (0-100%),

TiO₂ (0-100%),

ZrO₂ (0-100%).

The material may also be surface-functionalized. For example, the silanol groups may have been treated by silanization with silanes. The surface may be hydrophobized, or anionic and/or cationic groups and/or chelators may be applied. For example, a nitrilotriacetic acid (NTA) moiety may be applied as a chelator group. This allows the adsorber surface to be adapted to the biomolecules to be bound.

Another option is to apply halogen-containing atom transfer radical initiators to the silanol groups, using a silanization process, so that it is possible to produce polymer chains using a “grafting from” process at the silanol groups. This method, also referred to as graft copolymerization, requires polymerization processes in which the tendency toward chain termination, disproportionation, or recombination is slight. Initiators must be applied to the silanol groups in order to carry out a graft copolymerization. This may be achieved by treating the PECVD silicate layer with halogen-containing silanes. Alternatively, the initiators are introduced directly in the PECVD process. In this regard, volatile halogen-containing compounds are added to the process gas (precursor in the PECVD process). If an ATRP is carried out at such a halide-containing surface, polymers (homopolymers, copolymers, block copolymers) which are covalently bound to the surface are produced in situ. Suitable monomers are radically polymerizable compounds such as acrylates, methacrylates, styrene, and styrene derivatives, for example.

Atom transfer radical polymerization (ATRP) is a form of in situ radical polymerization. The radicals are formed from an organic halide via a Cu(I)/Cu(II) redox equilibrium, using an atom transfer process. The redox equilibrium results in a great reduction in the concentration of free radicals. Chain termination reactions due to disproportionation or recombination are thus greatly suppressed.

The term “amplification reaction” is understood to mean a method which allows the concentration of one or more analytes, preferably nucleic acids, to be at least doubled.

A distinction is made here between isothermal and thermocyclic amplification reactions. In the former, the temperature remains constant during the entire process, whereas in the latter, thermal cycles are run through which are used to control the reaction and the amplification.

The following are examples of preferred isothermal amplification reactions:
Loop mediated isothermal amplification (LAMP),
Nucleic acid sequence-based amplification (NASBA),
Rolling circle chain reaction (RCCR) or rolling circle amplification (RCA), and/or
Transcription mediated amplification (TMA).

The following are examples of preferred thermocyclic amplification reactions:
Ligase chain reaction (LCR), and/or
Polymerase chain reaction (PCR).

The term “polymerase chain reaction” (PCR) is understood to mean a method for in vitro amplification of nucleic acids, as described, for example, in Bartlett and Stirling (2003).

The term “ligase chain reaction” (LCR) is understood to mean a detection method for very small quantities of nucleic acids which functions similarly to the polymerase chain reaction, except that a different enzyme is used (a ligase instead of the polymerase). Two samples per DNA strand are ligated to form a sample. The resulting amplification products of a cycle, which are often only 30-50 by in length, are themselves reused in the subsequent cycles as a starting point for the supplemented primers.

The term “loop-mediated isothermal amplification” (LAMP) is understood to mean a method for isothermal nucleic acid amplification in which six different primers are used which recognize specific regions on the target sequence and bind thereto. LAMP uses a DNA polymerase having strand displacement activity, and proceeds at a constant temperature of approximately 65° C. Amplification and detection of the target sequence take place in a single step.

The term “nucleic acid sequence-based amplification” (NASBA) is understood to mean a method for amplifying RNA (Compton, 1991). An RNA matrix is added to a reaction mixture, and a first primer binds to the complementary sequence in the region of the 3′ end of the matrix. The DNA strand which is complementary to the matrix is then polymerized using a reverse transcriptase. The RNA matrix is then digested with the aid of RNase H (RNase H digests only RNA in RNA-DNA hybrids, but not single-stranded RNA). A second primer is then bound to the 5′ end of the DNA strand. This primer is used by the T7 RNA polymerase as the starting point for synthesizing an RNA molecule which is complementary to the DNA strand, and this RNA molecule may then be used once again as a starting matrix. NASBA is normally carried out at a constant temperature of 41° C., and under certain circumstances provides quicker and better results than PCR.

The term “transcription mediated amplification” (TMA) is understood to mean an isothermal amplification method, developed by the US company Gen-Probe, which is similar to NASBA and which likewise uses RNA polymerase and reverse transcriptase (Hill, 2001).

The term “rolling circle chain reaction” (RCCR) or “rolling circle amplification” (RCA) refers to an amplification method which imitates general nucleic acid replication according to the rolling circle principle, and which is described in U.S. Pat. No. 5,854,033, among other sources.

The term “immuno-PCR” (IPCR) is understood to mean in particular a method for detecting target molecules, in which chimeric conjugates from target-specific antibodies and nucleic acid molecules are used.

By their nature, the referenced target molecules primarily involve proteins and/or oligopeptides, since highly specific antibodies against this molecular species are most easily produced. However, the referenced target molecules may also involve other biomolecular species, for example oligo- and polysaccharides or lipids, provided that highly specific antibodies against this biomolecular species may be produced, so that the antibodies may be detected using immuno-PCR.

The nucleic acid molecules are used as markers or probes, which are amplified for signal generation using polymerase chain reaction (PCR). The very high efficiency of the nucleic acid amplification and the high specificity of the binding may result in a 100- to 10,000-fold increase in sensitivity compared to standard methods for detecting target molecules (ELISA method, for example). IPCR was developed in 1992 (Sano et al. (1992)).

The term “reverse transcription” is understood to mean a method for transcribing mRNA to DNA (so-called “cDNA”), in which a reverse transcriptase (also RNA-dependent DNA polymerase) is generally used. The latter first synthesizes an RNA-DNA hybrid strand from a single-strand RNA by means of RNA-dependent DNA polymerase activity. An independent portion of the protein, the RNase H portion, is responsible for the subsequent degradation of the RNA portion. The completion of the single-strand DNA strand to form double-strand DNA occurs by means of DNA-deposited DNA polymerase activity. The cDNA generated in this manner may then be amplified and detected using PCR.

The same as for other DNA polymerases, a reverse transcriptase also requires primers for initiating the DNA synthesis. In this case a so-called oligo d(T) primer, i.e., multiple thymine bases, is often used, which is complementary to the poly(A) tail at the 3′ end of the mRNA.

The combination of reverse transcription and subsequent PCR (also referred to as RT-PCR) is frequently used to detect the content of one or more mRNAs in a sample, for example in gene expression analysis, in the creation of gene expression profiles, and the like. In so-called “two-step RT-PCR,” among other things different primers are used for the reverse transcription and for the subsequent PCR, whereas in “one-step RT-PCR” the gene-specific primers used in the reverse transcription may also be used for the subsequent PCR, and the two reactions are conducted in succession in the same vessel. Use is made of the fact that the reverse transcriptase that is used (generally of viral origin) denatures at a lower temperature than the DNA polymerase that is used (Taq polymerase, for example), which, as is known, denatures only at relatively high temperatures, and accordingly the reverse transcription is carried out at a lower temperature than the subsequent PCR. A so-called “hot start” DNA polymerase which is thermoreversibly inhibited is preferably used. When a switch is made from the lower temperature level of the reverse transcription to the higher temperature level of the PCR, on the one hand the reverse transcriptase is denatured, and on the other hand the DNA polymerase is activated by eliminating the thermoreversible inhibition. The referenced thermoreversible inhibition may be achieved, for example, using an antibody which binds in the active center of the DNA polymerase, or, for example, also by reversible covalent or noncovalent chemical modification of the polymerase using aldehydes, for example (see, for example, U.S. Pat. No. 6,183,998 of the present applicant). For an overview, also see Birch et al. (1996).

The term “real-time PCR” is also understood to mean quantitative PCR or qPCR (not to be confused with reverse transcribed PCR), which is based on the principle of the known polymerase chain reaction (PCR), and which also allows the amplified DNA to be quantified. The quantification is performed by fluorescence measurements recorded during a PCR cycle (hence, the name “real time”). The fluorescence increases in proportion to the quantity of the PCR products. At the end of a run (composed of multiple cycles) the quantification is performed in the exponential phase of the PCR, based on the received fluorescence signals. Correct quantification is possible only in the exponential phase of the PCR (which lasts for a few cycles in a run), since the optimal reaction conditions are present during this phase. This method therefore differs from other quantitative PCR methods, which perform quantitative analysis only after the PCR (competitive PCR, for example) has proceeded, usually with inclusion of gel electrophoretic separation of the PCR fragments.

Stains such as ethidium bromide, SYBR green I, and FRET probes or so-called double-dye oligos (also referred to as TaqMan probes) are suitable for the detection.

The term “C_{T} value” (threshold cycle) refers to the PCR cycle for which an amplification product is first detectable; as a rule the fluorescence is measured, and the most recent cycle for which there was a significant increase above the background fluorescence for the first time is indicated.

In the initial phase of a PCR reaction the quantity of templates (i.e., on DNA to be amplified) is still limited, whereas in the final phase of the amplification the quantity of products increases in such a way that these products cause inhibition, and product fragments increasingly hybridize with one another, and the starting products are gradually consumed. Only in the phase in-between is there an exponential relationship between the number of amplification cycles and the quantity of amplification product (“exponential phase”). The referenced C_{T} value is used for determining the point in time at which the exponential phase begins.

Moreover, a low C_{T} value indicates that a small number of PCR cycles is sufficient for a first-time significant increase in the fluorescence above the background noise (i.e., a relatively large number of templates was present), whereas a high C_{T} value correspondingly indicates that a large number of PCR cycles is necessary for this purpose (i.e., relatively few templates were present).

The term “enzyme-linked immunosorbent assay” (ELISA) is understood to mean an immunological detection method which is based on an enzymatic color reaction.

By use of ELISA it is possible to detect proteins, viruses, and low-molecular compounds such as hormones, toxins, and pesticides in a sample (blood serum, milk, urine, etc.). In this regard, use is made of the property of specific antibodies of binding to the substance (antigen) to be detected. Antibodies or antigens are labeled beforehand with an enzyme. The reaction which is catalyzed by the enzyme is used as documentation of the presence of the antigen. The so-called substrate is converted by the enzyme, and the reaction product may usually be detected by a color change, fluorescence, or chemoluminescence. The signal strength is generally a function of the antigen concentration, so that ELISA may also be used for quantitative detection.

The term “hybrid capture assay” (HCA) below is understood to mean a method in which RNA-DNA hybrids are formed by incubation of the sought target DNA with an RNA sample. The hybrids are bound to a surface and then incubated with an enzyme-labeled antibody. Hybrid capture assays are used in particular in the HPV assays of the company Digene.

The term “nested PCR” below is understood to mean a method in which a DNA fragment which has already been amplified is amplified another time; this procedure is carried out using a second primer pair provided inside the primer pair used in the first reaction.

### OBJECT OF THE PRESENT INVENTION

The object of the present invention is to at least substantially overcome the described disadvantages resulting from the prior art, and to provide, in particular for a wide range of applications, a method, a use, and/or an apparatus for normalizing the content of biomolecules in a sample.

It is a particular object of the present invention to provide a method, a use, and/or an apparatus which is better suited for normalizing biomolecules in a sample, and which is appropriate for use with the above-mentioned one-step and two-step methods.

A further object is to provide for a wide range of applications a method, a use, and an apparatus for normalizing the content of a nucleic acid in a sample.

A further object is to provide for a wide range of applications a method, a use, and an apparatus for normalizing the content of a cDNA produced by reverse transcription from RNA, preferably mRNA.

A further object is to provide for a wide range of applications a method, a use, and an apparatus for normalizing the content of a nucleic acid, in particular RNA, in a sample.

It is an object of the present invention in particular to provide a method, a use, and/or an apparatus for normalizing biomolecules in a sample, which is/are characterized by high accuracy and reproducibility.

A further object is to provide a method, a use, and an apparatus for normalizing the content of a nucleic acid in a sample, and which allow multiple use of the reaction vessels.

These objects are achieved by a method according to the independent method claim of the present invention. Accordingly, a method [is provided] for normalizing the content of biomolecules in a sample, having the following steps:
a) Providing a reaction vessel having a vessel surface that is functionalized at least in places, preferably at the inside of the vessel, in such a way that the vessel surface is able to reversibly bind biomolecules under high salt conditions,
b) Carrying out at least one sample preparation step,
c) Binding biomolecules from the prepared sample to the vessel surface (“binding and normalizing step”) under high salt conditions,
d) Optionally washing (“washing step”), and
e) Carrying out at least one subsequent reaction.

In contrast to methods known from the prior art, in the present case the surface of the reaction vessel for the first time acts as an adsorption surface for the reversible binding of a defined quantity of biomolecules under high salt conditions, and therefore acts as a means for normalizing the biomolecule content in the sample. The surface of the reaction vessel is therefore functionalized in such a way that it is able to reversibly bind biomolecules under high salt conditions. The type of functionalization also depends in particular on the type of biomolecules to be bound. This will be discussed further below. The quantity of biomolecules which may be bound to the surface is adjusted by means of the size of the surface, the surface that is contacted by the sample, the type of chemical functionalization of this surface, the incubation time of the biomolecules with the surface, and the stringency of the binding buffer used.

Furthermore, the referenced high salt conditions (see definition above) assist in deactivating any RNases that are present. This ensures that, unlike the case for the low salt conditions that are present for charge-switch materials and in general for anion exchangers, RNA contained in a sample may be isolated in a substantially complete and intact manner and supplied for detection.

In the prior art, for the so-called “two-step method” as previously described, at least one additional pipetting step is necessary for transferring the isolated biomolecules into a new reaction vessel before a subsequent reaction, if needed, is carried out. This step is also used as an opportunity to normalize on the cDNA level. In this way fluctuations in the cellular input, the quality and quantity of RNA, and the RT efficiency are compensated for so that the results may be compared and interpreted.

In the one-step RT-PCR method, using the procedure proposed herein normalization would be performed on the RNA input level, since the cDNA is synthesized in situ and is directly reacted with the polymerase. In this way fluctuations in the cellular input and the quality and quantity of RNA may be compensated for.

The previously described disadvantages associated with these steps are not present for the method according to the invention. The biomolecules bind reversibly to the surface of the reaction vessel according to the invention until the surface according to the invention is saturated, resulting in normalization of the quantity of biomolecules. For this reason, in the method according to the invention no separate quantification step for the isolated quantity of biomolecules is necessary.

Furthermore, a subsequent reaction may be carried out directly in the reaction vessel according to the invention, without additional pipetting steps.

The normalization has the further advantage of correcting the expression data with regard to differences in the cellular input, RNA quality/quantity, and efficiency of the reverse transcription for various samples (the latter principle applies only for two-step RT-PCR).

It is preferably provided that the referenced subsequent reaction is carried out in the same vessel as the sample preparation step. However, this is not absolutely necessary. For example, after the binding and normalization step one or more aliquots may be withdrawn from the reaction vessel and transferred to one or more new reaction vessels in order to carry out the at least one subsequent reaction in the further reaction vessel(s).

It is particularly preferably provided that the referenced biomolecules are nucleic acids.

Nucleic acids may be detected in particular using conventional amplification methods such as PCR, for example.

However, the referenced biomolecules may in general also be any biomolecular species that is detectable using antibodies. Intended in particular are proteins that are detectable using oligonucleotide-labeled antibodies (“immuno-PCR”) or by ELISA (see below).

It is also preferably provided that the referenced at least one sample preparation step is selected from the group including
cell lysis,
cell denaturation,
isolation of biomolecules,
purification of biomolecules,
reverse transcription (RT) of RNA to DNA, and/or
enzymatic reactions and/or sample processing.

The referenced enzymatic reactions and/or sample processing may preferably involve digestion of a sample using RNases, DNases, and/or proteases.

It is also preferably provided that the referenced at least one subsequent reaction is selected from the group including
amplification reactions,
enzyme-linked immunoassay (ELISA), and/or
hybrid capture assay.

It is particularly preferably provided that the referenced amplification reaction is a reaction selected from the following group:
polymerase chain reaction (PCR),
nested PCR,
reverse transcription (RT),
immuno-PCR.

In general, however, any other possible detection reaction may be provided for at least one of the referenced biomolecular species.

One particularly suitable example of the method according to the invention is a method having reverse transcription of mRNA to cDNA (sample preparation step), normalization of the formed cDNA by binding to the vessel surface (binding and normalization step), and the subsequent detecting amplification of the cDNA by real-time PCR (subsequent reaction). This method is referred to in Table 1 as “Workflow 1.”

In such a method the reverse transcription is carried out directly in a reaction vessel according to the invention, and after incubation the generated cDNA is reversibly bound to the surface according to the invention. The surface is thus saturated, and it is possible to bind defined quantities of cDNA in this manner. Excess cDNA is removed during the subsequent washing step. After this washing step, the cDNA may be subjected to real-time PCR in the same reaction vessel.

The advantage of this method according to the invention compared to methods from the prior art is that neither quantification of the resulting quantity of cDNA nor an additional step of pipetting into a new reaction vessel is necessary after the reverse transcription. This results in a simplified or shortened protocol, and thus a significant decrease in the level of effort and/or the operating costs, as well as a reduction in the risk of cross-contamination and/or inaccuracy caused by the additional pipetting step.

A further advantage of this method according to the invention compared to methods from the prior art is that, in spite of eliminating quantification of the resulting quantity of cDNA, a normalized quantity of cDNA is used for the PCR, regardless of the quantity of RNA originally used. In addition, operations may be performed in a volume of 25 μL, which is customary for PCR, resulting in a significant cost reduction.

The reverse transcription as well as the subsequent PCR is preferably carried out in the same vessel, as described. However, it may also be provided that after the binding and normalization step one or more aliquots are withdrawn from the reaction vessel and transferred to one or more new reaction vessels in order to carry out the PCR in the further reaction vessel(s).

Another suitable example of the method according to the invention is a method having sample lysis and subsequent harvesting of mRNA, for example using the QIAGEN product RNeasy, or alternatively, with the isolation of mRNA, for example using the QIAGEN products Oligotex and/or TurboCapture (sample preparation step), normalization of the released or isolated mRNA by binding to the vessel surface (binding and normalization step), and the subsequent reverse transcription of the mRNA to cDNA (subsequent reaction). The reverse transcription may optionally be followed by an additional binding and normalization step and an additional subsequent reaction, for example real-time PCR of the produced cDNA (see Table 1, Workflow 6).

Alternatively, a simple hybridization reaction may be provided as the subsequent reaction; in this case the additional binding and normalization step may be omitted (see Table 1, Workflow 2).

Further examples of workflows for the method according to the invention are provided in Table 1. Workflows 1-5 involve a one-step method, since in the course of the method it is not necessary to open the reaction vessel to add new reagents. Workflow 6 shows a two-step method, since in this case normalization on the mRNA level and cDNA level is performed; i.e., the vessel is opened between the two reactions in order to initiate the second binding and normalization step.

TABLE 1

| Step | Workflow 1 | Workflow 2 | Workflow 3 | Workflow 4 | Workflow 5 | Workflow 6 |
|---|---|---|---|---|---|---|
| Sample | Reverse | Sample lysis and | Sample lysis and | Sample lysis and | Sample lysis and | Sample lysis and |
| preparation | transcription | harvesting of | harvesting of | harvesting of | harvesting of | harvesting of |
| step | (mRNA → cDNA) | mRNA | mRNA | DNA | biomolecules, e.g. | mRNA |
| | | | | | target proteins | |
| Binding and | Normalization | Normalization | Normalization | Normalization | Normalization | Normalization |
| normalization | of the | of the | of the | of the | of the | of the |
| step 1 | generated cDNA | harvested mRNA | harvested mRNA | harvested DNA | harvested | harvested mRNA |
| | | | | | biomolecules | |
| Subsequent | PCR | Hybridization with | Reverse transcription | PCR | Binding of oligo- | Reverse |
| reaction 1 | | labeled probes and | (mRNA → cDNA) | | labeled antibodies | transcription |
| | | detection | | | | (mRNA → cDNA) |
| Binding and | n/a | n/a | n/a | n/a | n/a | Normalization |
| normalization | | | | | | of the |
| step 2 | | | | | | generated cDNA |
| Subsequent | n/a | n/a | PCR | n/a | Immuno-PCR | PCR |
| reaction 2 | | | | | | |
| Normalization | cDNA level | mRNA level | mRNA level | DNA level | Biomolecular level, | mRNA level and |
| performed on: | | | | | e.g. target proteins | cDNA level |

The reverse transcription and the optionally following second subsequent reaction (in particular the PCR) may be carried out in the same vessel as the sample preparation step. However, it may also be provided that after the first or optionally the second binding and normalization step, one or more aliquots are withdrawn from the reaction vessel and transferred to one or more new reaction vessels in order to carry out the at least one subsequent reaction in the further reaction vessel(s).

For the referenced normalization step, for example 10 to 50 ng biomolecules (preferably RNA and/or DNA) could be isolated from the sample, depending on the size of the modified surface and the established binding conditions.

According to the invention, it is further provided that

a) a binding buffer is used in the binding and normalization step, and/or

b) a wash buffer is used in the washing step.

It is also preferably provided that the vessel surface, which is functionalized at least in places, contains
a) silanol groups,
b) unsaturated organic acids,
c) carboxyl groups, sulfonate groups, and other polar groups, and/or
d) metal oxides and semimetal oxides containing hydroxyl groups.

In contrast to the above-mentioned charge-switch materials, the referenced groups may consistently be permanently (i.e., generally covalently) bound to the surface of microreaction vessels and PCR vessels, using suitable methods (see below).

The binding of nucleic acids to silica matrices is known by the term “Boom principle,” and is described in EP 819696, for example (also see Vogelstein and Gillespie (1979) and Boom et al. (1990)).

For selective binding of nucleic acids from a sample, the sample is incubated in a buffer containing a chaotropic substance, for example guanidinium thiocyanate. The cells are optionally lysed, the obtained proteins are denatured, and the nucleic acids, if they are not yet freely available, are released, and the hydrate shells around the nucleic acids are dissolved due to the presence of the chaotropic substance. The nucleic acids are bound to the silica surface via hydrogen bridges between the silanol groups (SiOₓ or SiOH groups) of the silica matrix and the negative ion charges of the phosphate backbone of the nucleic acids. The remaining constituents of the sample may then be removed by washing. Lastly, the DNA or RNA is released under the conditions of the PCR.

The binding of the nucleic acids to an anion exchanger surface is based on the electrostatic interaction between the negative ion charge of the phosphate backbone of the nucleic acids and the positive surface charge of the anion exchanger surface according to the invention. Quaternary ammonium groups belong to the group of strongly basic anion exchangers, since their charge is independent of the pH of the binding buffer. Primary, secondary, and tertiary amines are referred to as weakly basic anion exchangers. At higher pH values these amines are present in deprotonated form, resulting in loss of the exchanger function. Thus, the exchange capability of weakly basic anion exchangers is greatly dependent on the pH of the binding buffer used.

Phenomena similar to those for silanol groups, in particular SiO₂, are also observed for metal oxides and semimetal oxides having hydroxyl groups at the surface, in particular titanium oxides, aluminum oxides, and zirconium oxides such as TiO₂, Al₂O₃, and ZrO₂, respectively. Nucleic acids also bind to these groups in the presence of chaotropic salts, and correspondingly coated or functionalized surfaces may likewise be used under the stated conditions in order to bind nucleic acids.

The referenced unsaturated organic acids must be polymerizable, i.e., must contain at least one unsaturated C=C bond (“vinylogous groups”), such as maleic acid, for example. In addition, they cannot be used in pure form, since otherwise they do not adhere to polypropylene, for example. Therefore, the unsaturated organic acids are applied to the surfaces in combination with vinyl silane, using the PECVD process, for example.

The unsaturated organic acids which are bound in this manner provide carboxyl groups (—COO⁻ or —COOH groups) which are able to reversibly bind nucleic acids under high salt conditions. In this regard the unsaturated organic acids are only the means to the end of introducing carboxyl groups into a PECVD-coated polymer in order to produce a polar surface having a hydrogen bridge donor functionality.

The solubility of nucleic acids in water is decreased by high salt [conditions]. The reason is the breaking down of hydrogen bridges and the associated decrease in the stability of secondary and tertiary structures of the nucleic acids in water. If a polar surface is then provided as a hydrogen bridge donor, the nucleic acids bind to this surface, since at that location they are more stable than in water. If the salt concentration is decreased, water once again becomes a better hydrogen bridge donor than the polar surface, and the nucleic acids may be detached from the surface.

Also provided according to the invention is a reaction vessel for carrying out a method as described above, having a vessel surface that is functionalized at least in places, preferably at the inside of the vessel, in such a way that the vessel surface is able to reversibly bind biomolecules under the referenced process conditions.

The region of the reaction vessels functionalized according to the invention preferably occupies a surface of 0.01 mm²-10 cm² per reaction vessel, particularly preferably a surface of 0.01 mm²-1 cm², very particularly preferably a surface of 0.01 mm²-500 mm², and even more preferably a surface of particularly preferably 0.01 mm², particularly preferably 100 mm². This means, for example, that a 96-well PCR plate, a PCR 8-strip, or a multititer plate has the referenced coated surface multiplied by the number of its wells.

The binding capacity of the reaction vessel in question for the biomolecules in question may be precisely set by selectively adjusting the characteristics (in particular the type and density of the functional groups) and the dimensions of the functionalized surface. Besides the surface, the quantity of biomolecules which is bound may also be influenced via the contact time and the stringency of the binding buffer. However, the objective is to achieve the normalization by completely saturating the provided surface in the reaction vessel with biomolecules.

It is preferably provided that the binding capacity per reaction vessel is in the range of 1 ng-40 μg, particularly preferably 1 ng-4 μg, more preferably 1 ng-2 μg, very particularly preferably 1 ng-500 ng.

Assuming a binding capacity of 50 ng per reaction vessel, for example, this means for the case that the sample in question, which has been subjected beforehand to lysis, cell denaturation, an isolation step, or reverse transcription, for example, contains greater than 50 ng of the biomolecular species in question, the excess fraction of biomolecules is not bound, and is removed in the subsequent washing step. Thus, the information concerning the quantitative fraction of the biomolecule(s) in question may possibly be lost. However, loss of the absolute quantitative information is tolerable for a majority of the possible application scenarios.

Conversely, this would mean that if the sample contains less than 50 ng of the biomolecular species in question, the binding capacity is not completely utilized, and therefore it is not possible to perform normalization.

This means that in practice there is an inclination to adjust the binding capacity in such a way (see above) that it tends to vary rather below or at the lower limit of the expected quantity of biomolecules in the sample.

It is preferably provided that the vessel surface, which is functionalized at least in places, contains
a) silanol groups,
b) unsaturated organic acids,
c) carboxyl groups, sulfonate groups, and other polar groups, and/or
d) metal oxides and semimetal oxides containing hydroxyl groups.

It is preferably provided that the above-referenced functional groups
a) are applied to the material of the reaction vessel by plasma coating,
b) are applied to the material of the reaction vessel by wet chemical methods, and/or
c) are dictated by the properties of the material of the reaction vessel itself.

The application of the silanol groups according to the invention by plasma coating onto the material of the reaction vessel according to the invention is preferably carried out in atmospheric pressure plasma, as described, for example, in DE 102006036536 B3 and DE 000010322696 B3 of the Fraunhofer Institute for Surface Engineering and Thin Films IST, to which reference is made for the entire contents thereof.

This method is also referred to as “plasma enhanced chemical vapor deposition” (PECVD). This method is a special form of chemical vapor deposition (CVD), in which thin layers are applied to a surface via plasma-assisted chemical reactions. For this purpose, in a reaction chamber an intense electrical field is applied between the substrate to be coated and a counter electrode, which causes ignition of a plasma. The plasma results in breakage of the bonds of a gaseous deposition medium, also referred to as reaction gas, and decomposes same into individual radicals which further react in the gas phase. The gas phase reaction products deposit onto the substrate in the form of thin layers (layer thicknesses between 50 and 300 nm). On account of the plasma, a higher deposition rate and at the same time a lower deposition temperature is achieved with the PECVD method, also referred to as corona discharge, than with the CVD method.

A basic requirement for the deposition of a given material is that this material must be capable of being provided in a gaseous aggregate state; this is often achieved using a so-called precursor, i.e., a compound which must have a certain vapor pressure at a given temperature, and which contains in a chemically bound form material which is to be deposited. In this manner, the deposition media to be used are already in the gas phase and may thus be easily introduced from the gas supply system, located outside the reaction chamber, into the reaction chamber and supplied to the plasma. Thus, “diamond like carbon” (DLC) or the carbon-containing gases acetylene (C₂H₂) or methane, for example, are used as precursor for producing a carbon-containing coating. Tetramethylsilane (TMS), tetraethoxysilane (TEOS), or tetramethoxysilane (TMOS), for example, are suitable for producing a silica coating. Additional suitable precursors exist for the deposition of TiO₂, Al₂O₃, and ZrO₂, for example.

The precursor is fed into the discharge zone of the plasma, where the gas is split into ions which are accelerated. Oxygen is often fed at the same time in order to combust a possible organic fraction in the precursor, for example for producing a silica coating using tetramethylsilane (not, however, for producing a carbon-containing coating using acetylene).

The gas ions then collide at high velocity with the surface of the workpiece to be coated, where they are reduced and form the coating in question. Covalent bonds are frequently formed between the material of the surface and the coating materials which ensure permanent binding of the coating to the material.

Thus, radicals result on the polypropylene (PP) in the plasma at a polypropylene surface due to homolytic splitting of C—C and C—H bonds. These radicals may form covalent bonds with the oxygen, silicon, or carbon of the coating material, for example.

The coating material is thus covalently bonded to the polypropylene surface. In the case of SiO₂ coatings with TEOS as precursor, the covalent bonding to PP occurs via Si—O—C and Si—C bonds. In the case of organic monomers as precursors, the covalent bonding to PP occurs via C—C and C—O—C bonds, for example.

Even when covalent bonds are not formed, however, extremely durable coatings are achieved in this manner.

The PECVD method also allows organic polymers to be generated in the gas phase in the plasma and deposited on the substrate. In this case, monomers such as maleic anhydride, acrylates, vinyl silanes, and other polymerizable precursors (monomers), for example, may be used. The oxygen may also be dispensed with in the carrier gas to prevent oxidation of the organic constituents.

However, this method may also be used to deposit silanol group-containing layers which directly result in anion exchanger layers. The precursor aminopropyltrimethoxysilane allows direct production of silanol groups and anion exchanger groups, for example, in a PECVD layer.

Silica layers containing carboxyl groups may also be produced by the selection of suitable precursors.

Carboxyl group-containing layers may also be produced on polypropylene using the precursors vinyltrimethoxysilane and maleic anhydride.

Metal oxides and semimetal oxides may likewise be produced, using the PECVD method, from the corresponding precursors (metal alkoxides and semimetal alkoxides) in the gas phase with addition of oxygen, and deposited on polypropylene, for example.

In addition to the metal alkoxides and semimetal alkoxides, gas phase polymers of acrylates, for example, and other unsaturated compounds may be produced and coated in situ via PECVD. By proper selection of the monomers (for example, HEMA, acrylic acid, maleic anhydride, etc.) and vinyl silane, mixed copolymers of an organic monomer such as maleic anhydride and a silane (vinyl silane) may be deposited on polypropylene. In this case, a polymer is produced which has carboxyl groups as well as silanol groups.

Hydroxyl groups (geminal, vicinal), diol groups, carboxyl groups, amino groups, and silanol groups in particular are suitable chemical functions for providing surface materials having hydrogen bridge donor properties on polypropylene.

Within the scope of the present invention, copolymers of maleic anhydride and vinyl silane have also been produced in situ. It is advantageous that this precursor mixture has a sufficient vapor pressure to be polymerizable in the gas phase via PECVD, and forms well-adhering layers on polypropylene, i.e., the material of the reaction vessel.

Styrenesulfonic acid, for example, may be used as a precursor for depositing sulfonate groups via PECVD.

In contrast to the above-mentioned CVD methods, in PECVD methods the temperature remains at approximately room temperature. PECVD methods are therefore also suited for the coating of plastics, for example polypropylene and polyethylene, which are frequently used for the reaction vessels according to the invention.

The inventors of the present invention have also demonstrated that, using a suitable coating apparatus, the inner surfaces of microreaction vessels, in particular PCR reaction vessels (“8-strips,” 96-well plates, multititer plates), disposable reaction vessels, and pipette tips may be coated using tetraethoxysilane and carboxyl-containing copolymers of vinyl silane and maleic anhydride as precursors. A corresponding apparatus is shown in FIG. 7.

The illustrated apparatus may also be used in a parallel configuration, thus allowing multiple reaction vessels to be coated at the same time.

Sol gel processes, for example, are preferred wet chemical methods for applying silanol groups to the material. The precursors together with a defined quantity of water and possible catalysts are dissolved in a solvent, for example water. Tetraethoxysilane (TEOS) is preferably used as silicon dioxide precursor.

However, it may be provided in particular that the silanol groups according to the invention are dictated by the properties of the material of the reaction vessel according to the invention itself, for example when the material is glass.

According to the invention, it is also preferably provided that the reaction vessel is a vessel from the group including
a) PCR vessels, PCR 8-strips, or PCR 96-well plates,
b) a capillary or a microfluid channel,
c) a disposable reaction vessel,
d) a pipette tip, and/or
e) a multititer plate.

The referenced microreaction vessels may be, for example, optionally sealable vessels, having a volume of 0.1-2 mL, which may also be referred to in colloquial usage as PCR reaction vessels (ABI, Thermo, etc.). These vessels are generally made of polypropylene, polyethylene, COC, PET, or polycarbonate.

This also similarly applies for the referenced pipette tips, which are generally used as disposable articles in conjunction with automatic pipettes (also often referred to in colloquial usage as “Eppendorf pipettes”).

A microtiter plate is a unit having a plurality of reaction vessels (“wells”) within the meaning of the invention. Such microtiter plates generally have 6 to 1536 wells. Typical microtiter plate formats are shown in Table 2.

TABLE 2

| | Number of reaction | | | |
|---|---|---|---|---|
| Type of | vessels (wells) | | Typical maximum filling | |
| vessel | per plate | Format | volume per well | |
| Microtiter | 6 | 2 × 3 | 2-5 | mL |
| plate | 12 | 3 × 4 | 2-4 | mL |
| | 24 | 4 × 6 | 0.5-3 | mL |
| | 96 | 8 × 12 | 0.3-2 | mL |
| | 384 | 16 × 24 | 0.03-0.1 | mL |
| | 1536 | 32 × 48 | 0.01 | mL |
| PCR soft strip | 8 | 1 × 8 | 0.2 | mL |
| (“8-strip”) | | | | |
| PCR 96-well | 96 | 8 × 12 | 0.2 | mL |
| plate | | | | |

In addition, a kit is provided for carrying out a method as described above, the kit having at least
a) a binding buffer,
b) a wash buffer,
c) optional reagents for carrying out a sample preparation step, preferably a reverse transcription (RT),
d) optional reagents for carrying out a subsequent reaction, preferably a polymerase chain reaction (PCR), and
e) optionally at least one reaction vessel according to the above description.

It is also preferably provided that the wash buffer contains water, Tris, a complexing agent, a polyol, a detergent, and a polymer, copolymer, and/or terpolymer.

It is also preferably provided that the binding buffer contains chaotropic substances. This is particularly preferably at least one substance selected from the group including
guanidine hydrochloride,
guanidinium (iso)thiocyanate,
sodium iodide,
potassium iodide,
sodium (iso)thiocyanate, and/or
urea,

or a mixture thereof.

The binding buffer for binding the cDNA to an anion exchanger surface is preferably a low salt buffer. A pH below the pKs value of the surface or of the surface groups is preferably set so that in the binding step the anion exchangers have positive surface charges, and are thus able to bind the negatively charged nucleic acids.

Also provided is the use of a reaction vessel according to the invention and/or a kit according to the invention for normalizing the content of biomolecules in a sample.

This preferably involves cDNA which has been produced in the reaction vessel according to the invention by reverse transcription (RT) from RNA, preferably mRNA, the cDNA then being subjected to a polymerase chain reaction (PCR).

Disclaimer

The above-mentioned components and the components claimed and described in the exemplary embodiments to be used according to the invention are not subject to any special exceptions with regard to their size, shape, material selection, or technical design, so that the selection criteria known in the field of application may have unlimited applicability. In addition, the following examples have no limiting effect on the scope of protection of the present patent application, which is defined solely on the basis of the claims.

### DRAWINGS AND EXAMPLES

Further particulars, features, and advantages of the subject matter of the invention result from the subclaims and from the following description of the associated figures and examples, in which multiple exemplary embodiments and fields of application of the present invention are illustrated by way of example.

FIG. 1 shows a diagram for checking the C_{T} values of PAXGene RNA from human whole blood for use in a reaction vessel according to the invention,

FIG. 2 shows a diagram for checking the C_{T} values of PAXGene RNA from human whole blood for use in a reaction vessel according to the invention, and comparative examples,

FIG. 3 shows a diagram for checking the C_{T} values of QIAamp RNA from human whole blood for use in a reaction vessel according to the invention, and comparative examples,

FIG. 4 shows a diagram for checking the C_{T} values of QIAamp RNA from

Jurkat cells for use in a reaction vessel according to the invention, and comparative examples,

FIG. 5 shows a diagram for checking the C_{T} values of QIAamp RNA from Jurkat cells for use in a reaction vessel according to the invention, and different incubation times,

FIG. 6 shows the general pattern of a workflow according to the invention, and

FIG. 7 shows an apparatus 70 for applying a surface, which is functionalized according to the invention, to the inside of a reaction vessel.

The present invention is explained in greater detail using the following exemplary embodiments, but is not intended to be limited thereto.

### EXAMPLE 1

The following procedure was carried out:

The surface-modified reaction vessels used according to the invention (PCR vessel, 0.2 mL, thin-walled, PCR soft strips, from Biozym) were coated with tetraethoxysilane (TEOS) in atmospheric pressure plasma.

PAXGene RNA and QIAamp RNA from human whole blood as well as QIAamp RNA from Jurkat cells were used in the conducted tests. The reverse transcription was carried out with the aid of the QIAGEN QuantiTect kit and the Omniscript kit, using a poly dT primer.

6 M GuHCl, 0.1 M potassium hydrogen phthalate (pH 2.5) was used as binding buffer, and 2% Nonidet® P40 and 0.1 mg/mL poly(methylvinyl ether-alt-maleic acid) in TE buffer was used as wash buffer. The PCR was carried out in an ABI 7700, using an ABI TaqMan® β-actin sample kit.

The test protocol is shown in Table 3:

TABLE 3

| No | Method step |
|---|---|
| 1. | Carrying out the reverse transcription according to manufacturer |
| | protocol in a silica-coated PCR vessel according to the invention |
| 2. | Addition of 50 μL binding buffer/vessel |
| 3. | Incubation for 20 min at room temperature |
| 4. | Complete removal of the liquid by aspiration |
| 5. | Addition of 120 μL wash buffer/vessel |
| 6. | Complete removal of the liquid by aspiration |
| 7. | Addition of 25 μL PCR mastermix and carrying out the PCR. |

The RNA used in the reverse transcription was quantified beforehand using a Nanodrop ND-1000 spectrophotometer.

As controls, in each case aliquots of the RT assays in uncoated PCR vessels were tested, and the aliquots of the RT reactions were pipetted directly into the PCR mastermix.

In each of the PCR experiments an eight-fold determination was performed; i.e., the stated values correspond to the average of eight individual values, and the error bars indicate the standard deviation.

### EXAMPLE 2

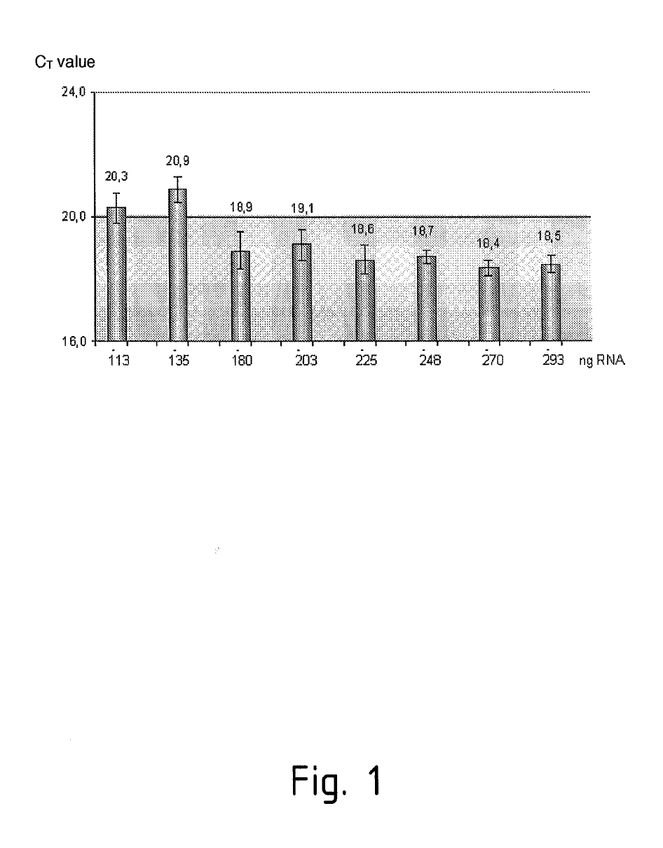

FIG. 1 shows the results of a TaqMan® run on the cDNA from a QuantiTect cDNA synthesis of PAXGene RNA from human whole blood. The quantity of RNA used was varied within a small range (113-293 ng).

The results surprisingly show that when a reaction vessel according to the invention is used, after the transition from 135 to 180 ng of RNA used a relatively uniform C_{T} level between 18.4 and 19.1, and therefore normalization of the C_{T} values, was achieved.

### EXAMPLE 3

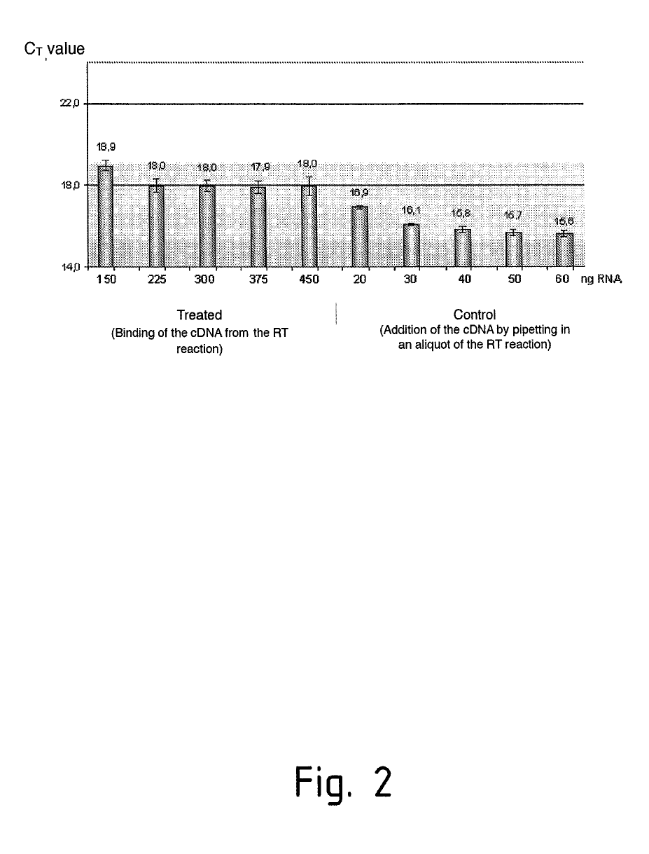

FIG. 2 likewise shows the results of a TaqMan® run on the cDNA from a QuantiTect cDNA synthesis of PAXGene RNA from human whole blood. The quantity of RNA was varied between 150 and 450 ng (columns 1-5), and in addition in each case an aliquot of the reverse transcription was pipetted into untreated vessels containing PCR mastermix (columns 6-10). For these aliquots the starting quantity of RNA contained therein is indicated. The quantity of RNA used varies over a wider range compared to the first example.

The results show that when a reaction vessel according to the invention is used, for use from 150 to 450 ng RNA a relatively uniform C_{T} level between 17.9 and 18.9, and therefore normalization of the C_{T} values, was likewise achieved. The control tests in untreated reaction vessels have shown that, as expected, the more RNA that was used as starting material for the reverse transcription, the greater was the decrease in the corresponding C_{T} values.

### EXAMPLE 4

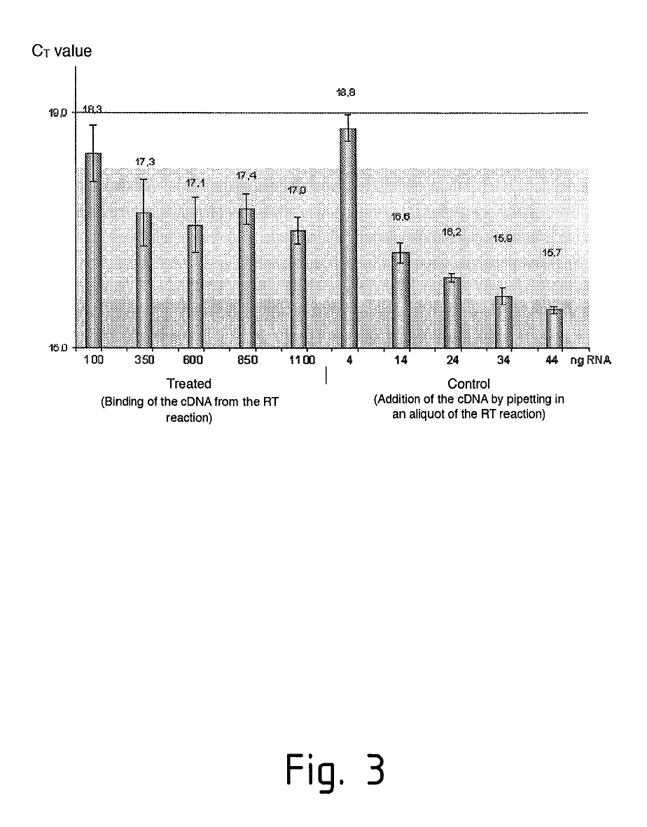

FIG. 3 shows the results of a TaqMan® run on the cDNA from an Omniscript cDNA synthesis of QIAamp RNA from human whole blood. The quantity of RNA was varied between 100 and 1100 ng RNA, and control tests were likewise carried out in uncoated vessels.

The results show that that when a reaction vessel according to the invention is used, for use from 100 to 1100 ng RNA a relatively uniform C_{T} level around 17.2 was present, and therefore normalization of the C_{T} values was likewise achieved. The control tests in untreated reaction vessels showed that, as expected, the more RNA that was used as starting material for the reverse transcription, the greater was the decrease in the corresponding C_{T} values.

### EXAMPLE 5

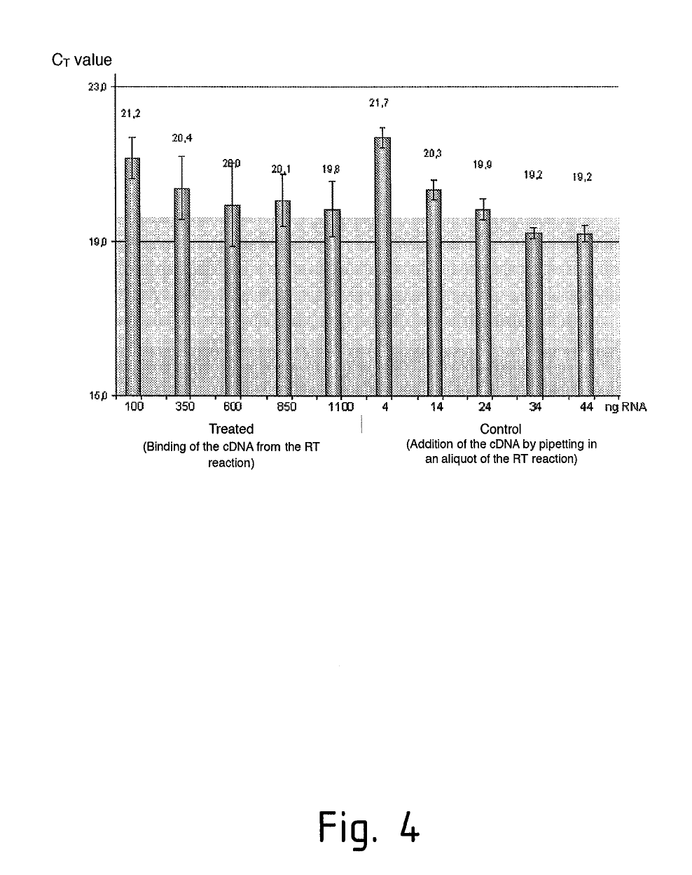

FIG. 4 shows the results of a TaqMan® run on the cDNA from an Omniscript cDNA synthesis of Jurkat RNA. The quantity of RNA was varied between 100 and 1100 ng RNA, and once again control tests were carried out in uncoated vessels.

The results show that that when a reaction vessel according to the invention is used, for use from 100 to 1100 ng RNA normalization of C_{T} values was achieved. The control tests in untreated reaction vessels once again showed that the more RNA that was used as starting material for the reverse transcription, the greater was the decrease in the corresponding C_{T} values.

### EXAMPLE 6

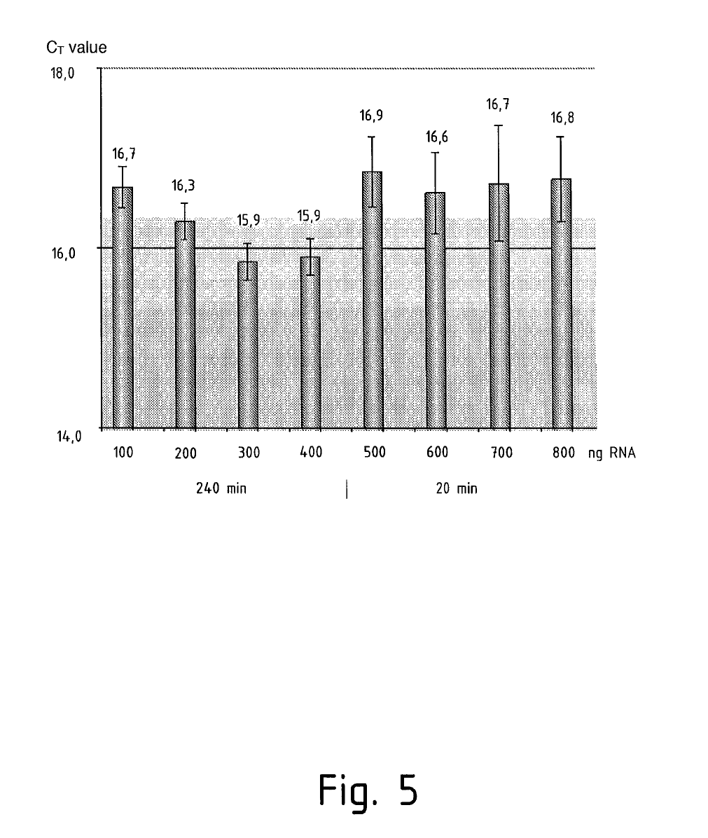

FIG. 5 shows the results of a TaqMan® run on the cDNA from an Omniscript cDNA synthesis of Jurkat RNA. The quantity of RNA was varied between 100 and 800 ng RNA. In addition, different incubation times were selected, namely, 240 min for 100-400 ng RNA, and 20 min for 500-800 ng RNA.

The results surprisingly show that by extending the incubation times from 20 to 240 min, it was possible to bring the C_{T} value for the lowest quantity of RNA (100 ng) to the level of saturation of the larger RNA quantities (500 to 800 ng), using a 20-minute incubation time. It is also apparent from this example that for average quantities of 300 to 400 ng RNA used and long incubation times of 240 min, saturation was achieved at a relatively low C_{T} level of 15.9.

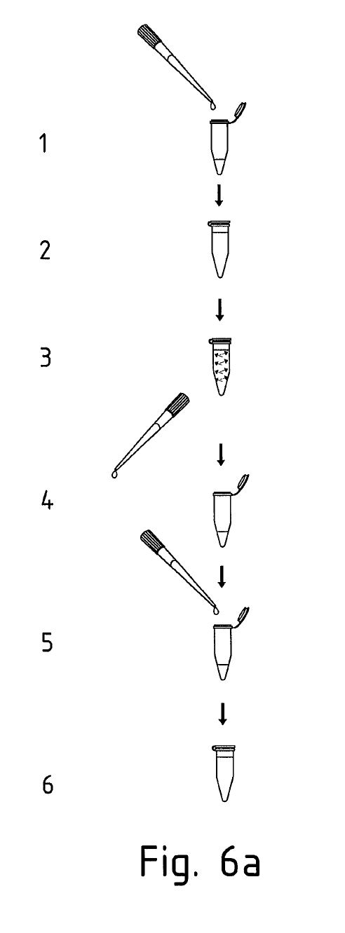

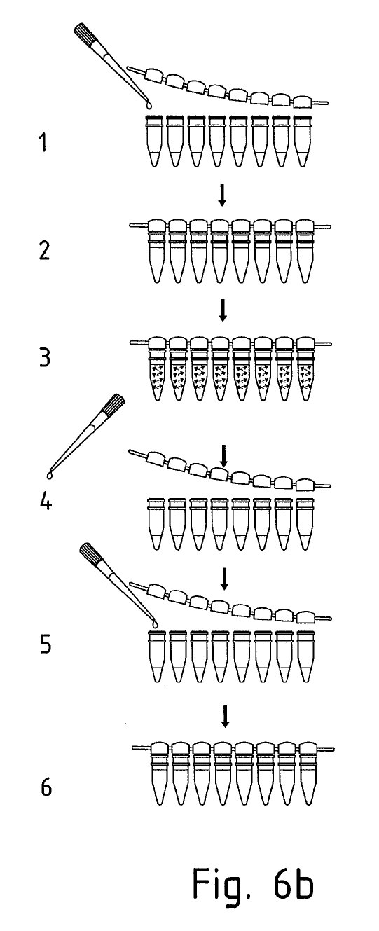

FIG. 6a shows the general pattern of a workflow according to the invention, including sample preparation step 2, binding and normalization step 3, washing step 4, and subsequent reaction 6. The subsequent reaction may optionally be followed by a second binding and normalization step and a second subsequent reaction (see Table 1).

It is shown in FIG. 6b that the referenced workflow may also be carried out in a PCR strip.

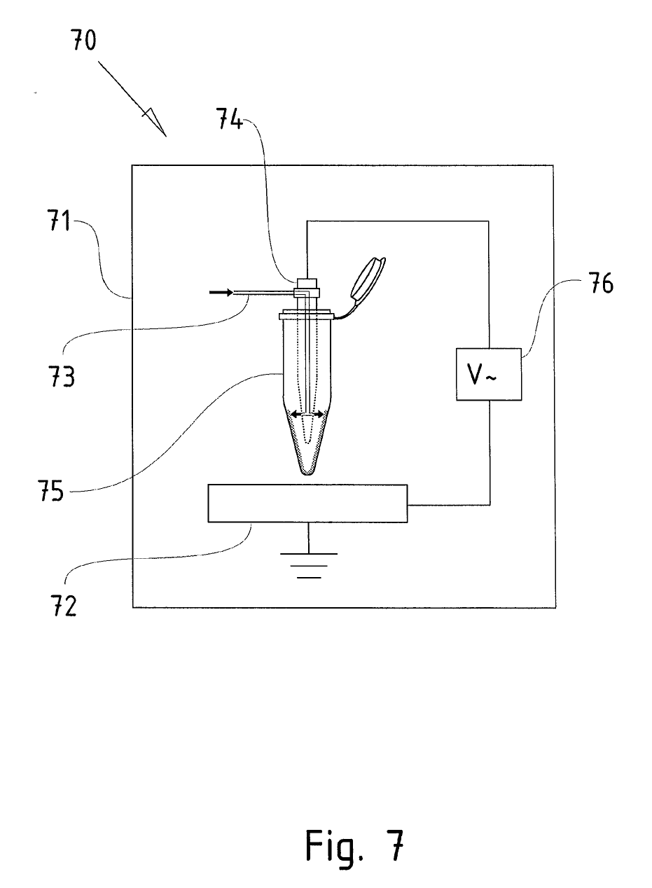

FIG. 7 shows an apparatus 70 for applying a surface, which is functionalized according to the invention, to the inside of a reaction vessel.

The referenced apparatus has a chamber 71 in which a flat electrode 72 is situated. The chamber also has a gas introduction device for a precursor gas 73, and a coating electrode 74. The precursor gas is tetraethoxysilane (TEOS), for example. The gas introduction device 73 and the coating electrode 74 are brought together in the apparatus according to FIG. 7 to form a combined device whose shape is adapted to the interior of a reaction vessel 75 to be coated (in the present case, a polypropylene PCR microreaction vessel having a volume of 0.2 mL, referred to in colloquial usage as an “Eppendorf vessel”).

A high-frequency alternating voltage (13.56 MHz, for example) is then applied between the flat electrode 72 and the coating electrode 74, using a frequency generator 76, and a plasma ignites inside the reaction vessel. The plasma results in breakage of the bonds of the precursor gas, and decomposes same into individual radicals which deposit on the substrate and at that location bring about the chemical deposition reaction of silica molecules.

When the reaction vessel functionalized according to the invention is used, the functionalized surface produced in this manner results in binding of the biomolecules to the inside of the reaction vessel (for example, the binding of nucleic acids to silanol groups on the functionalized surface in the presence of chaotropic salts).

The illustrated apparatus is also suitable for the simultaneous coating of multiple reaction vessels, such as multiple “Eppendorf vessels” or also a multititer plate having multiple wells, for example.

The size of the coated surface of the reaction vessels is essentially a function of the size of the electrode, and in a 0.2-mL PCR vessel may be 10 mm² to 300 mm².

### EXAMPLE 7

Polypropylene PCR vessels having a volume of 0.2 mL were coated with tetraethoxysilane, using the PECVD method under atmospheric pressure. Binding capacities of up to 500 ng for nucleic acids were achieved.

The surface produced during the coating is defined, among other factors, by the size and positioning of the electrodes (cathode, anode) with respect to one another for the PECVD method. In the present case, the size was approximately 150 mm².

### REFERENCES:

Bartlett & Stirling (2003), Methods Mol. Biol. 226:3-6
Sano et al. (1992), Science 258, 120-122
Birch et al. (1996), Nature 381, 445
Vogelstein and Gillespie (1979), PNAS 76: 615-619
Boom et al (1990), J. Clin. Microbiol. 1990 March ; 28(3):495-503
Compton (1991), Nature 350, 91
Hill (2001), Expert Reviews of Molecular Diagnostics 1, 445

1-17. (canceled)

18. A method for normalizing the content of biomolecules in a sample, comprising:
a) providing a reaction vessel having a vessel surface at least a portion of which is functionalized so that the vessel surface is able to reversibly bind biomolecules under high salt conditions,
b) carrying out at least one sample preparation step,
c) binding biomolecules from the prepared sample to the vessel surface under high salt conditions,
d) optionally washing the vessel surface of step c), and
e) carrying out at least one subsequent reaction.

19. The method of claim 18, wherein the portion of the reaction vessel that is functionalized is at the inside of the reaction vessel.

20. The method according to claim 18, wherein the biomolecules are nucleic acids.

21. The method according to claim 18, wherein the at least one sample preparation step is selected from the group consisting of cell lysis, cell denaturation, isolation of biomolecules, purification of biomolecules, reverse transcription of RNA to DNA, and enzymatic reactions or sample processing.

22. The method according to claim 18, wherein the at least one subsequent reaction is selected from the group consisting of amplification reactions, enzyme-linked immunoassay (ELISA), and hybrid capture assay.

23. The method according to claim 22, wherein the amplification reaction is a reaction selected from the group consisting of polymerase chain reaction (PCR), reverse transcription (RT), loop mediated isothermal amplification (LAMP), nucleic acid sequence-based amplification (NASBA), rolling circle chain reaction (RCCR) or rolling circle amplification (RCA), transcription mediated amplification (TMA), ligase chain reaction (LCR), nested PCR, and immuno-PCR.

24. The method according to claim 18, wherein step b) comprises reverse transcription (RT) from RNA to generate cDNA, step c) comprises binding the cDNA to the vessel surface under high salt conditions, and step e) comprises performing a polymerase chain reaction (PCR) using the cDNA as a template.

25. The method according to claim 18, wherein
a) a binding buffer is used in step c), or
b) a wash buffer is used in step d).

26. The method according to claim 18, wherein
a) a binding buffer is used in step c), and
b) a wash buffer is used in step d).

27. The method according to claim 18, wherein the portion of the vessel surface that is functionalized comprises one or more groups selected from (a) silanol groups, (b) unsaturated organic acids, (c) carboxyl groups, sulfonate groups, or other polar groups, and (d) metal oxides containing hydroxyl groups or semimetal oxides containing hydroxyl groups.

28. The method according to claim 25, wherein the wash buffer comprises one or more reagents selected from water, Tris, a complexing agent, a polyol, a detergent, a polymer, copolymer, and terpolymer.

29. The method according to claim 26, wherein the wash buffer comprises one or more reagents selected from water, Tris, a complexing agent, a polyol, a detergent, a polymer, copolymer, and terpolymer.

30. The method according to claim 25, wherein the binding buffer comprises a chaotropic substance.

31. The method according to claim 26, wherein the binding buffer comprises a chaotropic substance.

32. The method according to claim 30, wherein the chaotropic substance is at least one substance selected from the group consisting of guanidine hydrochloride, guanidinium (iso)thiocyanate, sodium iodide, potassium iodide, sodium (iso)thiocyanate, urea, and mixtures thereof

33. The method according to claim 31, wherein the chaotropic substance is at least one substance selected from the group consisting of guanidine hydrochloride, guanidinium (iso)thiocyanate, sodium iodide, potassium iodide, sodium (iso)thiocyanate, urea, and mixtures thereof

34. A reaction vessel, wherein at least a portion of the surface of the reaction vessel is functionalized so that the vessel surface is able to reversibly bind biomolecules under high salt conditions.

35. The reaction vessel according to claim 34, wherein the portion of the vessel surface that is functionalized comprises one or more groups selected from (a) silanol groups, (b) unsaturated organic acids, (c) carboxyl groups, sulfonate groups, or other polar groups, and (d) metal oxides containing hydroxyl groups, or semimetal oxides containing hydroxyl groups.

36. The reaction vessel according to claim 34, wherein the function groups are:
(a) applied to the material of the reaction vessel by plasma coating,
(b) applied to the material of the reaction vessel by wet chemical methods, or
(c) dictated by the properties of the material of the reaction vessel itself

37. A kit, comprising:
a binding buffer,
a wash buffer,
optional reagents for carrying out a sample preparation step,
optional reagents for carrying out a subsequent reaction, and
one or more reaction vessels having a vessel surface at least a portion of which is functionalized so that the vessel surface is able to reversibly bind biomolecules under high salt conditions.

38. The kit according to claim 37, wherein the sample preparation step is a reverse transcription (RT).

39. The kit according to claim 37, wherein the subsequent reaction is a polymerase chain reaction (PCR).

40. The kit according to claim 37, wherein the wash buffer comprises one or more reagents selected from water, Tris, a complexing agent, a polyol, a detergent, a polymer, copolymer, and terpolymer.

41. The kit according to claim 40, wherein the binding buffer comprises a chaotropic substance.

42. The kit according to claim 41, wherein the chaotropic substance is at least one substance selected from the group consisting of guanidine hydrochloride, guanidinium (iso)thiocyanate, sodium iodide, potassium iodide, sodium (iso)thiocyanate, urea, and mixtures thereof

43. A method for detecting biomolecules, comprising:
a) providing a reaction vessel having a vessel surface at least a portion of which is functionalized so that the vessel surface is able to reversibly bind biomolecules under high salt conditions,
b) carrying out at least one sample preparation step,
c) binding biomolecules from the prepared sample to the vessel surface under high salt conditions,
d) optionally washing the vessel surface of step c), and
e) carrying out a reaction that detects the biomolecules.

44. The method of claim 43, wherein the biomolecules are nucleic acids.

45. The method of claim 43, wherein the biomolecules are RNA.
